(19) United States
(12) Patent Application Publication
(10) Pub. No.: US 2011/0312095 A1
(43) Pub. Date: Dec. 22, 2011

(54) METHOD AND CONSTRUCTS FOR INCREASING RECOMBINANT PROTEIN PRODUCTION IN PLANTS DEHYDRATION STRESS

(75) Inventors: Serge Laberge, Sainte Foy (CA); Souad Azelmat, Sainte Foy (CA); Marc-Andre D'Aoust, Sainte Foy (CA); Pierre-Olivier Lavoie, Sainte Foy (CA); Jean Cloutier, Sainte Foy (CA); Yves Castonguay, Sainte Foy (CA); Louis-Philippe Vezina, Sainte Foy (CA)

(73) Assignee: Her Majesty the Queen in Right of Canada as Represented by the Minister of Agriculture

(21) Appl. No.: 12/811,903

(22) PCT Filed: Jan. 9, 2009
(86) PCT No.: PCT/IB09/05019

§ 371 Date: Jan. 28, 2011

Related U.S. Application Data
(60) Provisional application No. 61/006,369, filed on Jan. 9, 2008.

(51) Int. Cl. C12N 15/82; C12N 15/113

(52) U.S. Cl. 435/468; 536/24.1; 435/320.1

(57) ABSTRACT

The present invention provides methods and constructs for increasing recombinant protein production in plants during dehydration stress. The invention includes nucleic acids, inducible expression systems, and methods for using same for increasing recombinant protein production in plants and plant protection.

### CROSS-REFERENCE TO RELATED PATENT APPLICATIONS

This application claims priority from Provisional Application U.S. Application 61/006,369, filed Jan. 9, 2008, incorporated herein by reference in its entirety.

### FIELD OF THE INVENTION

The present invention provides methods and constructs for increasing recombinant protein production in plants during dehydration stress. Specifically, the invention provides an inducible expression system and methods for using same for increasing recombinant protein production in plants.

### BACKGROUND OF THE INVENTION

Traditional systems for producing pharmaceuticals, cosmetics, or compounds of agro-alimentary interest are based on microbial fermentation and cultured mammalian cells. The use of transgenic plants as bioreactors provides an alternative and valuable resource for producing recombinant molecules at lower cost and under safe conditions. One major limiting factor in the progress of this technology is the inability of transgenic organisms to accumulate adequate amounts of the recombinant product. To overcome this limitation, transcription rates of a gene of interest can be optimized, thus increasing the accumulation of mRNA and translational product.

Presently, constitutive promoters, such as the cauliflower mosaic virus (CaMV) 35S promoter and its derivatives, are commonly used for expressing genes in a host plant. However, constitutive expression systems may have limited utility under certain circumstances. For example, constitutive promoter systems may not be advantageous for expressing biologically active molecules because the universal expression of some recombinant proteins may detrimentally affect plant growth and development. Additionally, constitutive synthesis of foreign proteins is costly for the host plant in terms of metabolic resources and energy reserves. Moreover, constitutive promoters have low efficiency in several host plants, such as alfalfa.

In addition to the need for more efficient promoters, there is also a need for promoters that are responsive to abiotic and biotic conditions. For example, many temperate plants exposed to low, but non-freezing, temperatures increase their freezing tolerance. This adaptive process, called cold acclimation, is associated with biochemical and physiological alterations, as well as changes in gene expression. Thomashow M. F. Annu. Rev. Plant Physiol. 50: 571-599 (1999). Cold adaptive genes, referred to as COR (cold regulated) genes, have been cloned from many plant species, including Arabidopsis, alfalfa, and wheat. Thomashow M. F. Plant Physiol. 118: 1-7 (1998) (and references therein). Gilmour S. J. et al. Plant Mol. Biol. 18: 13-21 (1992). Laberge S. et al. Plant Physiol 101: 1411-1412 (1993). Houde M. et al. Plant Physiol. 99: 1381-1387 (1992). To date, only a few COR genes encode proteins with known functions, such as transcription factors, chaperones, enzymes required for osmolyte biosynthesis, and proteins involved in signal transduction. Fowler S, and Thomashow M. F. Plant Cell 14: 1675-1690 (2000); Seki M. et al. Plant J. 31: 279-292 (2002). While the precise function of the majority of the COR genes remains unknown, they are thought to encode, for example, LEA (late embryogenesis abundant) proteins, which accumulate in seeds during the later stages of embryogenesis, and in vegetative tissues in response to stress leading to water deficit such as cold, drought, salinity and ABA application. Ingram J. and Bartels D. Annu. Rev. Plant Physiol. Plant Mol. Biol. 47: 377-403 (1996).

The promoters of several cold responsive genes have been isolated. The best-studied cold-inducible promoters in dicotyledonous plants are the Arabidopsis rd29A and COR15A gene promoters (Yamaguchi-Shinozaki, K. and Shinozaki, K. Plant Cell 6: 251-264 (1994)); Baker, S. S., et al. Plant Mol. Biol. 24: 701-713 (1994)), and the Brassica napus gene BN115 (Jiang C., et al. Plant Mol. Biol. 30: 679-684 (1996)). The COR15A gene encodes a polypeptide that shares biochemical characteristics with dehydrins

Dehydrin proteins, which accumulate during cold acclimation in numerous herbaceous and woody plants, have been speculated to provide, among other things, protection from desiccative extracellular ice formation. In many plants, dehydrin proteins (group 2 late embryogenesis proteins (LEA D-11 family)) are induced by conditions that affect plant water status such as desiccation, salinity stress, and freezing stress. Cross, T. J., et al. Plant Mol Biol 23: 279-286 (1993). Low-temperature regulation of dehydrin genes involves one major cis-acting element, named DRE (dehydration responsive element) and also known as CRT (C-repeat) element. DRE mediates transcription in response to low temperatures, dessication, drought, or salinity, but not in response to ABA. Yamaguchi-Shinozaki K. and Shinozaki K. Trends Plant Science 10: 88-94 (2005).

Expression cassettes with regulatory elements from cold-inducible genes are used for expressing recombinant proteins in prokaryotic and eukaryotic expression systems. For example, U.S. Pat. No. 6,479,260 discloses an E. coli cold-inducible expression cassette based on the promoter of the cspA gene. Likewise, U.S. Pat. No. 6,084,089 discloses a promoter for cold inducible expression in potato tubers. Similarly, U.S. Pat. No. 6,184,443 discloses a cold-sensitive promoter for the expression of recombinant proteins in plant roots and tubers.

Expression cassettes have also been developed that direct protein expression specifically in foliar tissues. For example, the BN115 promoter (U.S. Pat. No. 5,847,102) targets protein expression to the leaves. Although the WCS120 promoter (U.S. Pat. No. 6,627,793) induces recombinant protein expression in alfalfa leaves (Ouellet F. et al. FEBS Lett. 423: 324-328 (1998)), the promoter's efficiency is unknown. Furthermore, the BN115 and WCS120 promoters were obtained from Brassica napus and wheat, respectively, and it is a goal of the present invention to develop a highly efficient expression cassette entirely derived from endogenous genomic sequences due to public concern about introducing foreign DNA into crop plants. That is, the present invention contemplates transformation strategies which maximize the re-introduction of endogenous DNA i.e. DNA cloned from the same plant species or close relatives. Rommens, C. et al. Plant Physiol. 135 (1):421-31 (2004).

Another aspect of regulating protein production involves protease control. Proteins are extracted at 4° C. in order to prevent degradation, as proteases activity is greatly inhibited by low temperatures. In planta, proteolysis is required for protein turnover and protease inhibitors can control proteolytic enzymes. While it has been shown that the expression of two Kunitz type protease inhibitors, BnD22 from rapeseed and WCP from cauliflower, are induced by drought and salinity (Downing W. L., et al. Plant J. 2: 685-693 (1992)); Nishio N. and Satoh H. Plant Physiol. 115: 841-846 (1997)), it is unclear whether these inhibitors protect proteins induced by water deficit. Thus, protease activity could represent an additional control point for regulating gene expression during dehydration stresses, including cold.

Accordingly, there is a need for an efficient, inducible expression system for expressing proteins in plant leaves based on native, plant genomic elements, such as those from alfalfa. The present invention provides an inducible expression system and methods for using same for increasing recombinant protein production in plants during dehydration stress.

### SUMMARY OF THE INVENTION

In one aspect, the invention provides a nucleic acid sequence isolated from alfalfa that increases expression of an operably linked gene in response to cold stress, salt stress, dehydration, or abscisic acid.

In one embodiment, the nucleic acid sequence is selected from the group consisting of SEQ ID NO: 1-2, or a variant thereof. In a further embodiment, the variant has a sequence identity that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% in sequence to SEQ ID NO: 1-2, and confers dehydrin promoter activity.

In another aspect, the invention provides an expression cassette comprising a promoter and terminator sequence isolated from alfalfa that increases expression of an operably linked gene in response to cold stress, salt stress, dehydration, or abscisic acid.

In one embodiment, the promoter is SEQ ID NO: 1 and the terminator is SEQ ID NO: 2. In another embodiment, a plant cell comprises the expression cassette. In another embodiment, a transgenic plant comprises a plant cell comprising the expression cassette. In another embodiment, a pharmaceutical, nutriceutical, or cosmetic is produced from a transgenic plant comprising a plant cell comprising the expression cassette.

In another aspect, the invention provides a method for enhancing the expression of a desired gene in a plant during low temperature stress, comprising (i) isolating a dehydrin promoter sequence from alfalfa; (ii) operably linking the promoter to the desired gene and a dehydrin terminator sequence; (iii) inserting the resulting cassette between T-DNA borders; and (iv) stably transforming the plant with said cassette.

In one aspect, the invention provides a method for enhancing the expression of a desired gene in a plant during low temperature stress, comprising (i) isolating a dehydrin promoter sequence from alfalfa; (ii) operably linking the promoter to the desired gene and a dehydrin terminator sequence; (iii) inserting the resulting cassette between T-DNA borders; and (iv) transiently transforming the plant with said cassette.

In another aspect, the invention provides a strong and inducible plant recombinant protein expression system, wherein said expression system comprises an alfalfa dehydrin promoter sequence operably linked to a desired gene and a dehydrin terminator sequence.

In another aspect, the invention provides a cold-inducible plant expression cassette, comprising alfalfa dehydrin promoter and terminator sequences.

In another aspect, the invention provides an Abscisic Acid (ABA)-responsive plant expression cassette, comprising an alfalfa dehydrin promoter sequence.

In another aspect, the invention provides a dehydration-inducible plant expression cassette, comprising alfalfa dehydrin promoter and terminator sequences.

In another aspect, the invention provides a method for plant protection, comprising (i) isolating a dehydrin promoter sequence from alfalfa; (ii) operably linking the promoter to the desired gene and a terminator; (iii) inserting the resulting cassette between T-DNA borders; and (iv) stably transforming said crop plant with said cassette.

In one embodiment, the promoter is SEQ ID NO: 1. In another embodiment, the terminator is SEQ ID NO: 2. In one embodiment, the plant is alfalfa.

In another aspect, the invention provides a method for inducing recombinant protein accumulation in a plant, comprising (i) isolating a dehydrin promoter sequence from alfalfa; (ii) operably linking the promoter to a desired gene and a terminator; (iii) inserting the resulting cassette between T-DNA borders; and (iv) stably transforming said plant with said cassette.

In another aspect, the invention provides a method for increasing recombinant protein accumulation in a plant during dehydration stress, comprising (i) isolating a dehydrin promoter sequence from alfalfa; (ii) operably linking the promoter to a desired gene and a terminator; (iii) inserting the resulting cassette between T-DNA borders; and (iv) stably transforming said plant with said cassette.

In another aspect, the invention provides a method for protecting an agronomically important crop plant from cold stress, drought stress, or salt stress, comprising (i) isolating a dehydrin promoter sequence from alfalfa; (ii) operably linking the promoter to a desired gene and a terminator; (iii) inserting the resulting cassette between T-DNA borders; and (iv) stably transforming said crop plant with said cassette.

In another aspect, the invention provides an expression cassette for increasing recombinant protein accumulation in a plant during dehydration stress, comprising a dehydrin promoter and terminator. In one embodiment, a plant cell comprises the expression cassette. In another embodiment, a transgenic plant comprises the expression cassette. In one embodiment, the terminator is a dehydrin terminator. In a further embodiment, the terminator is SEQ ID NO: 2.

In one aspect, the invention provides an alfalfa regulatory element that increases gene expression during dehydration stress. In one embodiment, the element is selected from the group consisting of a dehydrin promoter or dehydrin terminator sequence. In another embodiment, the element is set forth in SEQ ID NO: 1-2, or a variant thereof. In a further embodiment, the variant has a sequence identity that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% in sequence to SEQ ID NO: 1-2, and confers dehydrin promoter activity.

### BRIEF DESCRIPTION OF THE DRAWINGS

FIG. 1. Dot-blot analysis of dehydrin transcript accumulation in leaves of alfalfa after acclimation under the following conditions: Non-hardened (NH); Hardened two weeks at 2° C. (H2); Hardened two weeks at 2° C. followed by two weeks at −2° C. (H2F2).

FIG. 2. Southern blot analysis of the dehydrin genes in alfalfa plants. Genomic DNA was digested with HindIII (H), and DraI (D).

FIG. 3. Nucleic acid sequence of the 1.4 kb genomic insert containing the alfalfa dehydrin promoter. Primers used for the upstream walking in the secondary nested PCR are double underlined. The arrow indicates the beginning of the dehydrin promoter. The transcription start site was not determined and position +1 is assigned to the first nucleotide of the translation start codon (ATG) (shown in bold letters). The positions of the putative TATA box (italic printing) and ABRE-like motifs (single underline) are indicated. The two LTRE elements are boxed.

FIG. 4. Nucleic acid sequence of the 3′-flanking region of the alfalfa dehydrin gene. The gene specific primer GSP2 (DH2-3′) used for genome walking towards terminator is underlined. A codon stop is shown in bold letters and the location of extension primers used for the amplification of 847 bp fragment used in expression cassettes is indicated (double underline).

FIG. 5. Schematic representation of the expression cassettes. DH-P: dehydrin promoter. DH-T: dehydrin terminator. NOS: nos terminator. 2×35S-P: 2×35S promoter. TEV: translational enhancing leader sequence from the tobacco etch virus. HC-C5-1: Heavy chain of the C5-1 antibody. LC-C5-1: Light chain of the C5-1 antibody.

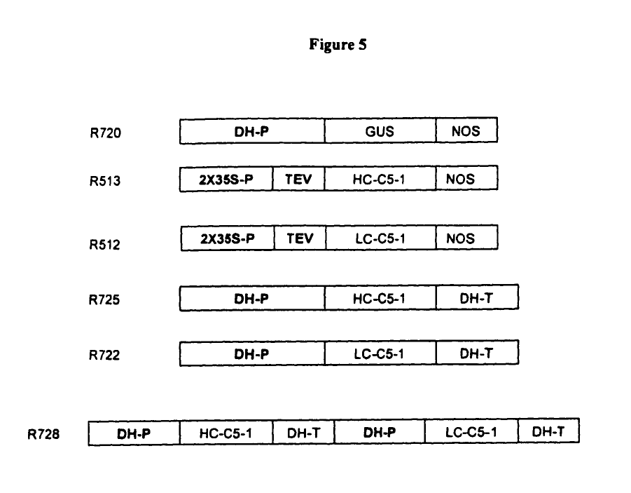

FIG. 6. Comparative analysis of antibody (C5-1) accumulation in ago-infiltrated leaves using the cold induced alfalfa dehydrin and the constitutive 2×35S/TEV promoters. Data are presented as the means ±SE of at least 4 independent extracts for each treatment. C5-1 expression was determined by Elisa. Control leaves were incubated for 4 days at 23° C. (4). Treated leaves were incubated for 3 days at 23° C. and then transferred for 14 days at 2° C. for cold acclimation (3-14).

FIG. 7. Protein blot analysis of C5-1 assembly in infiltrated alfalfa leaves during cold acclimation. Proteins were extracted form leaves co-infiltrated with expression cassettes R722+R725 or R512+R513. Control leaves were incubated for 4 days at 23° C. (4). Treated leaves were exposed to cold during 14 days after being incubated 3 days at 23° C. (3-14). Polyclonal anti-mouse IgG was used for detection. Two nanograms of purified C5-1 spiked in total soluble proteins extract from alfalfa leaves infiltrated with an empty vector was loaded as a control of detection.

FIG. 8. Quantification of C5-1 accumulation in agro-infiltrated alfalfa leaves. Control leaves were incubated at 23° C. for 4 days. Treated leaves were exposed to low temperatures (2° C.) during 7 days after 3 days incubation at 23° C. (3-7).

FIG. 9. Quantification of C5-1 accumulation in alfalfa leaves agro-infiltrated with pR728. Leaves incubated for 4 days at 23° C. were used as control. Treated leaves were allowed to acclimate for 4 days at 2° C. after 3 days at 23° C. (3-4).

FIG. 10. Effect of cold treatment on C5-1 accumulation in leaves from transgenic alfalfa. Detached leaves from 12 independent transgenic lines harbouring R728 construct were exposed to low temperatures (2° C.) during 7 and 14 days. The level of C5-1 was determined by Elisa using a standard curve generated with mouse IgG1 (Sigma, Cat. # M9269).

### DETAILED DESCRIPTION OF THE PREFERRED EMBODIMENTS

The present inventors realized the need for a plant inducible expression system and methods for using same for increasing recombinant protein production in plants, particularly during dehydration stress. Specifically, the invention provides an inducible expression system having plant-derived genetic elements, such as an alfalfa dehydrin promoter and terminator. To this end, nucleic acid constructs are provided having an alfalfa dehydrin promoter and terminator operably linked to a desired gene. These nucleic acid constructs may be used, for example, as a means for regulating protein expression during cold, drought, or salt stress. Additionally, the present constructs can be used to protect a plant from cold, drought, or salt stress.

The present invention uses terms and phrases that are well known to those practicing the art. Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described herein are those well known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. The techniques and procedures are generally performed according to conventional methodology (Molecular Cloning, A Laboratory Manual, 3rd. edition, edited by Sambrook & Russel Cold Spring Harbor Laboratory Press, Cold Spring Harbor, N.Y., 2001).

Agrobacterium or bacterial transformation: as is well known in the field, Agrobacteria that are used for transforming plant cells are disarmed and virulent derivatives of, usually, Agrobacterium tumefaciens, Agrobacterium rhizogenes, that contain a vector. The vector typically contains a desired polynucleotide that is located between the borders of a T-DNA. However, any bacteria capable of transforming a plant cell may be used, such as, Rhizobium trifolii, Rhizobium leguminosarum, Phyllobacterium myrsinacearum, SinoRhizobium meliloti, and MesoRhizobium loti.

Angiosperm: vascular plants having seeds enclosed in an ovary. Angiosperms are seed plants that produce flowers that bear fruits. Angiosperms are divided into dicotyledonous and monocotyledonous plants.

Aprotinin: is a protease inhibitor, also referred to as “bovine pancreatic trypsin inhibitor,” affects known serine proteases such as trypsin, chymotrypsin, plasmin and kallikrein.

Dehydration Stress refers to a condition where a plant cell undergoes substantial water loss. In the present invention, dehydration may result from, for example, cold or freezing temperature exposure, drought, pathogen infection, disease, herbivore attack, salt stress, hormonal signaling, high temperature or light exposure, changes in cuticular wax production, changes in stomatal aperture, water loss due to increased transpiration rate, root rot, or any other biochemical, physiological, or environmental stress resulting in water loss.

Dehydrin: Dehydrin proteins accumulate during cold temperature exposure in numerous herbaceous and woody plants, and they are thought to help acclimate a plant to low temperatures. Among other things, dehydrins protect a plant from desiccative extracellular ice formation. In many plants, dehydrins are induced by conditions that affect plant water status, such as desiccation, salinity stress, and freezing stress. Cross, T. J., et al. Plant Mol Biol 23: 279-286 (1993).

Dehydrin promoter activity: As used herein, dehydrin promoter activity refers to the ability of a promoter sequence to induce increased expression of an operably linked gene during or following dehydration stress. Dehydration stress can result from water deficit, salinity stress, and cold temperature exposure. For example, a cold-stressed plant having a dehydrin promoter operably linked to a gene encoding a C5-1 antibody would have increased levels of C5-1 protein expression, compared with a cold-stressed plant having a control promoter operably linked to the C5-1 coding sequence. Thus, the increased C5-1 expression is attributed to dehydrin promoter activity.

Desired Polynucleotide: a desired polynucleotide of the present invention is a genetic element, such as a promoter, enhancer, or terminator, or gene or polynucleotide that is to be transcribed and/or translated in a transformed cell that comprises the desired polynucleotide in its genome. If the desired polynucleotide comprises a sequence encoding a protein product, the coding region may be operably linked to regulatory elements, such as to a promoter and a terminator, that bring about expression of an associated messenger RNA transcript and/or a protein product encoded by the desired polynucleotide. Thus, a “desired polynucleotide” may comprise a gene that is operably linked in the 5′- to 3′-orientation, a promoter, a gene that encodes a protein, and a terminator. Alternatively, the desired polynucleotide may comprise a gene or fragment thereof, in a “sense” or “antisense” orientation, the transcription of which produces nucleic acids that may affect expression of an endogenous gene in the plant cell. A desired polynucleotide may also yield upon transcription a double-stranded RNA product upon that initiates RNA interference of a gene to which the desired polynucleotide is associated. A desired polynucleotide of the present invention may be positioned within a T-DNA, such that the left and right T-DNA border sequences flank or are on either side of the desired polynucleotide. The present invention envisions the stable integration of one or more desired polynucleotides into the genome of at least one plant cell. A desired polynucleotide may be mutated or a variant of its wild-type sequence. It is understood that all or part of the desired polynucleotide can be integrated into the genome of a plant. It also is understood that the term “desired polynucleotide” encompasses one or more of such polynucleotides. Thus, a T-DNA of the present invention may comprise one, two, three, four, five, six, seven, eight, nine, ten, or more desired polynucleotides.

Dicotyledonous plant (dicot): a flowering plant whose embryos have two seed halves or cotyledons, branching leaf veins, and flower parts in multiples of four or five. Examples of dicots include but are not limited to, Eucalyptus, Populus, Liquidamber, Acacia, teak, mahogany, cotton, tobacco, Arabidopsis, tomato, potato sugar beet, broccoli, cassava, sweet potato, pepper, poinsettia, bean, alfalfa, soybean, carrot, strawberry, lettuce, oak, maple, walnut, rose, mint, squash, daisy, geranium, avocado, and cactus.

Endogenous: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Foreign: “foreign,” with respect to a nucleic acid, means that that nucleic acid is derived from non-plant organisms, or derived from a plant that is not the same species as the plant to be transformed or is not derived from a plant that is not interfertile with the plant to be transformed, does not belong to the species of the target plant. According to the present invention, foreign DNA or RNA represents nucleic acids that are naturally occurring in the genetic makeup of fungi, bacteria, viruses, mammals, fish or birds, but are not naturally occurring in the plant that is to be transformed. Thus, a foreign nucleic acid is one that encodes, for instance, a polypeptide that is not naturally produced by the transformed plant. A foreign nucleic acid does not have to encode a protein product.

Gene: A gene is a segment of a DNA molecule that contains all the information required for synthesis of a product, polypeptide chain or RNA molecule that includes both coding and non-coding sequences.

Genetic element: a “genetic element” is any discreet nucleotide sequence such as, but not limited to, a promoter, gene, terminator, intron, enhancer, spacer, 5′-untranslated region, 3′-untranslated region, or recombinase recognition site.

Genetic modification: stable introduction of DNA into the genome of certain organisms by applying methods in molecular and cell biology.

Gymnosperm: as used herein, refers to a seed plant that bears seed without ovaries. Examples of gymnosperms include conifers, cycads, ginkgos, and ephedras.

Introduction: as used herein, refers to the insertion of a nucleic acid sequence into a cell, by methods including infection, transfection, transformation or transduction.

Monocotyledonous plant (monocot): a flowering plant having embryos with one cotyledon or seed leaf, parallel leaf veins, and flower parts in multiples of three. Examples of monocots include, but are not limited to turfgrass, maize, rice, oat, wheat, barley, sorghum, orchid, iris, lily, onion, and palm. Examples of turfgrass include, but are not limited to Agrostis spp. (bentgrass species including colonial bentgrass and creeping bentgrasses), Poa pratensis (kentucky bluegrass), Lolium spp. (ryegrass species including annual ryegrass and perennial ryegrass), Festuca arundinacea (tall fescue) Festuca rubra commutata (fine fescue), Cynodon dactylon (common bermudagrass varieties including Tifgreen, Tifway II, and Santa Ana, as well as hybrids thereof); Pennisetum clandestinum (kikuyugrass), Stenotaphrum secundatum (st. augustinegrass), Zoysia japonica (zoysiagrass), and Dichondra micrantha.

Native: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Operably linked: combining two or more molecules in such a fashion that in combination they function properly in a plant cell. For instance, a promoter is operably linked to a structural gene when the promoter controls transcription of the structural gene.

Phenotype: phenotype is a distinguishing feature or characteristic of a plant, which may be altered according to the present invention by integrating one or more “desired polynucleotides” and/or screenable/selectable markers into the genome of at least one plant cell of a transformed plant. The “desired polynucleotide(s)” and/or markers may confer a change in the phenotype of a transformed plant, by modifying any one of a number of genetic, molecular, biochemical, physiological, morphological, or agronomic characteristics or properties of the transformed plant cell or plant as a whole. Thus, expression of one or more, stably integrated desired polynucleotide(s) in a plant genome, may yield a phenotype selected from the group consisting of, but not limited to, increased drought tolerance, enhanced cold and frost tolerance, improved vigor, enhanced color, enhanced health and nutritional characteristics, improved storage, enhanced yield, enhanced salt tolerance, enhanced heavy metal tolerance, increased disease tolerance, increased insect tolerance, increased water-stress tolerance, enhanced sweetness, improved vigor, improved taste, improved texture, decreased phosphate content, increased germination, increased micronutrient uptake, improved starch composition, and improved flower longevity.

Plant Protection: refers to a condition whereby a plant displays drought tolerance; enhanced cold and frost tolerance; resistance to microbial, fungal, or insect pests; enhanced salt tolerance; enhanced heavy metal tolerance; increased pathogen and disease tolerance; increased insect tolerance, increased water-stress tolerance; or increased resistance to any herbivore.

Plant protection may be manifested by expression of defense genes, signaling compounds, phytoalexin production, reinforced cell walls, increased cuticular wax production, changes in stomatal aperture, improved vigor, enhanced growth and/or photosynthetic rate, enhanced color, enhanced health and nutritional characteristics, improved storage, enhanced yield, enhanced sweetness, improved taste, improved texture, decreased phosphate content, increased germination, increased micronutrient uptake, improved starch composition, and improved flower longevity.

Plant tissue: a “plant” is any of various photosynthetic, eukaryotic, multicellular organisms of the kingdom Plantae characteristically producing embryos, containing chloroplasts, and having cellulose cell walls. A part of a plant, i.e., a “plant tissue” may be treated according to the methods of the present invention to produce a transgenic plant. Many suitable plant tissues can be transformed according to the present invention and include, but are not limited to, somatic embryos, pollen, leaves, stems, calli, stolons, microtubers, and shoots. Thus, the present invention envisions the transformation of angiosperm and gymnosperm plants such as turfgrass, wheat, maize, rice, barley, oat, sugar beet, potato, tomato, tobacco, alfalfa, lettuce, carrot, strawberry, cassava, sweet potato, geranium, soybean, oak, pine, fir, acacia, eucalyptus, walnut, and palm. According to the present invention “plant tissue” also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed. Of particular interest are conifers such as pine, fir and spruce, monocots such as Kentucky bluegrass, creeping bentgrass, maize, and wheat, and dicots such as cotton, tomato, lettuce, Arabidopsis, tobacco, and geranium.

Plant transformation and cell culture: broadly refers to the process by which plant cells are genetically modified and transferred to an appropriate plant culture medium for maintenance, further growth, and/or further development. Such methods are well known to the skilled artisan.

Progeny: a “progeny” of the present invention, such as the progeny of a transgenic plant, is one that is born of, begotten by, or derived from a plant or the transgenic plant. Thus, a “progeny” plant, i.e., an “F1” generation plant is an offspring or a descendant of the transgenic plant produced by the inventive methods. A progeny of a transgenic plant may contain in at least one, some, or all of its cell genomes, the desired polynucleotide that was integrated into a cell of the parent transgenic plant by the methods described herein. Thus, the desired polynucleotide is “transmitted” or “inherited” by the progeny plant. The desired polynucleotide that is so inherited in the progeny plant may reside within a T-DNA construct, which also is inherited by the progeny plant from its parent. The term “progeny” as used herein, also may be considered to be the offspring or descendants of a group of plants.

Promoter: promoter is intended to mean a nucleic acid, preferably DNA that binds RNA polymerase and/or other transcription regulatory elements. As with any promoter, the promoters of the current invention will facilitate or control the transcription of DNA or RNA to generate an mRNA molecule from a nucleic acid molecule that is operably linked to the promoter. As stated earlier, the RNA generated may code for a protein or polypeptide or may code for an RNA interfering, or antisense molecule.

A plant promoter is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as xylem, leaves, roots, or seeds. Such promoters are referred to as tissue-preferred promoters. Promoters which initiate transcription only in certain tissues are referred to as tissue-specific promoters. A cell type-specific promoter primarily drives expression in certain cell types in one or more organs, for example, vascular cells in roots or leaves. An inducible or repressible promoter is a promoter which is under environmental control. Examples of conditions that may effect transcription by inducible promoters include cold temperature, dehydration, Abscisic Acid (ABA), and salt stress. Tissue specific, tissue preferred, cell type specific, and inducible promoters constitute the class of non-constitutive promoters. A constitutive promoter is a promoter which is active under most environmental conditions, and in most plant parts.

Polynucleotide is a nucleotide sequence, comprising a gene coding sequence or a fragment thereof, (comprising at least 15 consecutive nucleotides, preferably at least 30 consecutive nucleotides, and more preferably at least 50 consecutive nucleotides), a promoter, an intron, an enhancer region, a polyadenylation site, a translation initiation site, 5′ or 3′ untranslated regions, a reporter gene, a selectable marker or the like. The polynucleotide may comprise single stranded or double stranded DNA or RNA. The polynucleotide may comprise modified bases or a modified backbone. The polynucleotide may be genomic, an RNA transcript (such as an mRNA) or a processed nucleotide sequence (such as a cDNA). The polynucleotide may comprise a sequence in either sense or antisense orientations.

An isolated polynucleotide is a polynucleotide sequence that is not in its native state, e.g., the polynucleotide is comprised of a nucleotide sequence not found in nature or the polynucleotide is separated from nucleotide sequences with which it typically is in proximity or is next to nucleotide sequences with which it typically is not in proximity.

Seed: a “seed” may be regarded as a ripened plant ovule containing an embryo, and a propagative part of a plant, as a tuber or spore. Seed may be incubated prior to Agrobacterium-mediated transformation, in the dark, for instance, to facilitate germination. Seed also may be sterilized prior to incubation, such as by brief treatment with bleach. The resultant seedling can then be exposed to a desired strain of Agrobacterium.

Selectable/screenable marker: a gene that, if expressed in plants or plant tissues, makes it possible to distinguish them from other plants or plant tissues that do not express that gene. Screening procedures may require assays for expression of proteins encoded by the screenable marker gene. Examples of selectable markers include the neomycin phosphotransferase (NPTII) gene encoding kanamycin and geneticin resistance, the hygromycin phosphotransferase (HPT or APHIV) gene encoding resistance to hygromycin, or other similar genes known in the art.

Sequence identity: as used herein, “sequence identity” or “identity” in the context of two nucleic acid or polypeptide sequences includes reference to the residues in the two sequences which are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Sequence identity” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS IN MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in GUIDE TO HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215: 403 (1990)), and FASTDB (Brutlag et al., Comp. App. Biosci. 6: 237 (1990)).

Transcriptional terminators: The expression DNA constructs of the present invention typically have a transcriptional termination region at the opposite end from the transcription initiation regulatory region. The transcriptional termination region may be selected, for stability of the mRNA to enhance expression and/or for the addition of polyadenylation tails added to the gene transcription product. Translation of a nascent polypeptide undergoes termination when any of the three chain-termination codons enters the A site on the ribosome. Translation termination codons are UAA, UAG, and UGA.

In the present invention, it is preferable to use a native terminator that increases gene expression/stability during or following dehydration stress. SEQ ID NO: 2 is illustrative of a such a promoter.

Transfer DNA (T-DNA): an Agrobacterium T-DNA is a genetic element that is well-known as an element capable of integrating a nucleotide sequence contained within its borders into another genome. In this respect, a T-DNA is flanked, typically, by two “border” sequences. A desired polynucleotide of the present invention and a selectable marker may be positioned between the left border-like sequence and the right border-like sequence of a T-DNA. The desired polynucleotide and selectable marker contained within the T-DNA may be operably linked to a variety of different, plant-specific (i.e., native), or foreign nucleic acids, like promoter and terminator regulatory elements that facilitate its expression, i.e., transcription and/or translation of the DNA sequence encoded by the desired polynucleotide or selectable marker.

Transformation of plant cells: A process by which a nucleic acid is stably inserted into the genome of a plant cell. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols such as ‘refined transformation’ or ‘precise breeding’, viral infection, whiskers, electroporation, microinjection, polyethylene glycol-treatment, heat shock, lipofection and particle bombardment.

Transgenic plant: a transgenic plant of the present invention is one that comprises at least one cell genome in which an exogenous nucleic acid has been stably integrated. According to the present invention, a transgenic plant is a plant that comprises only one genetically modified cell and cell genome, or is a plant that comprises some genetically modified cells, or is a plant in which all of the cells are genetically modified. A transgenic plant of the present invention may be one that comprises expression of the desired polynucleotide, i.e., the exogenous nucleic acid, in only certain parts of the plant. Thus, a transgenic plant may contain only genetically modified cells in certain parts of its structure.

Variant: a “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. “Variant” may also refer to a “shuffled gene” such as those described in Maxygen-assigned patents.

It is understood that the present invention is not limited to the particular methodology, protocols, vectors, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a gene” is a reference to one or more genes and includes equivalents thereof known to those skilled in the art and so forth. Indeed, one skilled in the art can use the methods described herein to express any native gene (known presently or subsequently) in plant host systems.

### Polynucleotide Sequences

The present invention relates to an isolated nucleic molecule comprising a polynucleotide having a sequence set forth in any of SEQ ID NO: 1-2. The invention also provides functional fragments of the polynucleotide sequences of SEQ ID NO: 1-2. The invention further provides complementary nucleic acids, or fragments thereof, to SEQ ID NO: 1-2, as well as a nucleic acid, comprising at least 15 contiguous bases, which hybridizes to SEQ ID NO: 1-2.

By “isolated” nucleic acid molecule(s) is intended a nucleic acid molecule, DNA or RNA, which has been removed from its native environment. For example, recombinant DNA molecules contained in a DNA construct are considered isolated for the purposes of the present invention. Further examples of isolated DNA molecules include recombinant DNA molecules maintained in heterologous host cells or purified (partially or substantially) DNA molecules in solution. Isolated RNA molecules include in vitro RNA transcripts of the DNA molecules of the present invention. Isolated nucleic acid molecules, according to the present invention, further include such molecules produced synthetically.

Nucleic acid molecules of the present invention may be in the form of RNA, such as mRNA, or in the form of DNA, including, for instance, cDNA and genomic DNA obtained by cloning or produced synthetically. The DNA or RNA may be double-stranded or single-stranded. Single-stranded DNA may be the coding strand, also known as the sense strand, or it may be the non-coding strand, also referred to as the anti-sense strand.

Unless otherwise indicated, all nucleotide sequences determined by sequencing a DNA molecule herein were determined using an automated DNA sequencer (such as the Model 373 from Applied Biosystems, Inc.). Therefore, as is known in the art for any DNA sequence determined by this automated approach, any nucleotide sequence determined herein may contain some errors. Nucleotide sequences determined by automation are typically at least about 95% identical, more typically at least about 96% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence may be completely different from the amino acid sequence actually encoded by the sequenced DNA molecule, beginning at the point of such an insertion or deletion.

Each “nucleotide sequence” set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U) where each thymidine deoxynucleotide (T) in the specified deoxynucleotide sequence in is replaced by the ribonucleotide uridine (U).

The present invention is also directed to fragments of the isolated nucleic acid molecules described herein. Preferably, DNA fragments comprise at least 15 nucleotides, and more preferably at least 20 nucleotides, still more preferably at least 30 nucleotides in length, which are useful as diagnostic probes and primers. Of course larger nucleic acid fragments of up to the entire length of the nucleic acid molecules of the present invention are also useful diagnostically as probes, according to conventional hybridization techniques, or as primers for amplification of a target sequence by the polymerase chain reaction (PCR), as described, for instance, in Molecular Cloning, A Laboratory Manual, 3rd. edition, edited by Sambrook & Russel., (2001), Cold Spring Harbor Laboratory Press, the entire disclosure of which is hereby incorporated herein by reference. By a fragment at least 20 nucleotides in length, for example, is intended fragments which include 20 or more contiguous bases from the nucleotide sequence of SEQ ID NO: 1-2. The nucleic acids containing the nucleotide sequences listed in SEQ ID NO: 1-2 can be generated using conventional methods of DNA synthesis which will be routine to the skilled artisan. For example, restriction endonuclease cleavage or shearing by sonication could easily be used to generate fragments of various sizes. Alternatively, the DNA fragments of the present invention could be generated synthetically according to known techniques.

In another aspect, the invention provides an isolated nucleic acid molecule comprising a polynucleotide which hybridizes under stringent hybridization conditions to a portion of the polynucleotide in a nucleic acid molecule of the invention described above. By a polynucleotide which hybridizes to a “portion” of a polynucleotide is intended a polynucleotide (either DNA or RNA) hybridizing to at least about 15 nucleotides, and more preferably at least about 20 nucleotides, and still more preferably at least about 30 nucleotides, and even more preferably more than 30 nucleotides of the reference polynucleotide. These fragments that hybridize to the reference fragments are useful as diagnostic probes and primers. For the purpose of the invention, two sequences hybridize when they form a double-stranded complex in a hybridization solution of 6×SSC, 0.5% SDS, 5×Denhardt's solution and 100 μg of non-specific carrier DNA. See Ausubel et al., section 2.9, supplement 27 (1994). Sequences may hybridize at “moderate stringency,” which is defined as a temperature of 60° C. in a hybridization solution of 6×SSC, 0.5% SDS, 5×Denhardt's solution and 100 μg of non-specific carrier DNA. For “high stringency” hybridization, the temperature is increased to 68° C. Following the moderate stringency hybridization reaction, the nucleotides are washed in a solution of 2×SSC plus 0.05% SDS for five times at room temperature, with subsequent washes with 0.1×SSC plus 0.1% SDS at 60° C. for 1 h. For high stringency, the wash temperature is increased to 68° C. For the purpose of the invention, hybridized nucleotides are those that are detected using 1 ng of a radiolabeled probe having a specific radioactivity of 10,000 cpm/ng, where the hybridized nucleotides are clearly visible following exposure to X-ray film at −70° C. for no more than 72 hours.

The present application is directed to such nucleic acid molecules which are at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% identical to a nucleic acid sequence described in of SEQ ID NO: 1-2. Preferred, however, are nucleic acid molecules which are at least 95%, 96%, 97%, 98%, 99% or 100% identical to the nucleic acid sequence shown in of SEQ ID NO: 1-2. Differences between two nucleic acid sequences may occur at the 5′ or 3′terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 95%, 96%, 97%, 98% or 99% identical to a reference nucleotide sequence refers to a comparison made between two molecules using standard algorithms well known in the art and can be determined conventionally using publicly available computer programs such as the BLASTN algorithm. See Altschul et al., Nucleic Acids Res. 25:3389-3402 (1997).

### Sequence Analysis

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

The following running parameters are preferred for determination of alignments and similarities using BLASTN that contribute to the E values and percentage identity for polynucleotide sequences: Unix running command: blastall -p blastn -d embldb -e 10-G0-E0 -r 1 -v 30 -b 30 -i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (blastn only) [Integer]; -v Number of one-line descriptions (V) [Integer]; -b Number of alignments to show (B) [Integer]; -i Query File [File In]; and -o BLAST report Output File [File Out] Optional.

The “hits” to one or more database sequences by a queried sequence produced by BLASTN, FASTA, BLASTP or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, FASTA and BLASTP algorithms also produce “Expect” values for alignments. The Expect value (E) indicates the number of hits one can “expect” to see over a certain number of contiguous sequences by chance when searching a database of a certain size. The Expect value is used as a significance threshold for determining whether the hit to a database, such as the preferred EMBL database, indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the polynucleotide sequences then have a probability of 90% of being the same. For sequences having an E value of 0.01 or less over aligned and matched portions, the probability of finding a match by chance in the EMBL database is 1% or less using the BLASTN or FASTA algorithm.

According to one embodiment, “variant” polynucleotides, with reference to each of the polynucleotides of the present invention, preferably comprise sequences having the same number or fewer nucleic acids than each of the polynucleotides of the present invention and producing an E value of 0.01 or less when compared to the polynucleotide of the present invention. That is, a variant polynucleotide is any sequence that has at least a 99% probability of being the same as the polynucleotide of the present invention, measured as having an E value of 0.01 or less using the BLASTN, FASTA, or BLASTP algorithms set at parameters described above.

Alternatively, variant polynucleotides of the present invention hybridize to the polynucleotide sequence of SEQ ID NO: 1-2, or complements, reverse sequences, or reverse complements of those sequences, under stringent conditions.

The present invention also encompasses polynucleotides that differ from the disclosed sequences but that, as a consequence of the degeneracy of the genetic code, encode a polypeptide which is the same as that encoded by a polynucleotide of the present invention. Thus, polynucleotides comprising sequences that differ from the polynucleotide sequences recited in SEQ ID NO: 1-2, or complements, reverse sequences, or reverse complements thereof, as a result of conservative substitutions are contemplated by and encompassed within the present invention. Additionally, polynucleotides comprising sequences that differ from the polynucleotide sequences recited in SEQ ID NO: 1-2, or complements, reverse complements or reverse sequences thereof, as a result of deletions and/or insertions totaling less than 10% of the total sequence length are also contemplated by and encompassed within the present invention.

In addition to having a specified percentage identity to an inventive polynucleotide sequence, variant polynucleotides preferably have additional structure and/or functional features in common with the inventive polynucleotide. In addition to sharing a high degree of similarity in their primary structure to polynucleotides of the present invention, polynucleotides having a specified degree of identity to, or capable of hybridizing to an inventive polynucleotide preferably have at least one of the following features: (i) they contain an open reading frame or partial open reading frame encoding a polypeptide having substantially the same functional properties as the polypeptide encoded by the inventive polynucleotide; or (ii) they have domains in common. For example, a variant sequence could have structural similarity to SEQ ID NO: 1-2, or a variant may have dehydrin promoter activity.

### Source of Elements and DNA Sequences

Any or all of the elements and DNA sequences that are described herein may be endogenous to one or more plant genomes. Accordingly, in one particular embodiment of the present invention, all of the elements and DNA sequences, which are selected for the ultimate transfer cassette are endogenous to, or native to, the genome of the plant that is to be transformed. For instance, all of the sequences may come from an alfalfa genome. Alternatively, one or more of the elements or DNA sequences may be endogenous to a plant genome that is not the same as the species of the plant to be transformed, but which function in any event in the host plant cell. The present invention also encompasses use of one or more genetic elements from a plant that is interfertile with the plant that is to be transformed.

In this regard, a “plant” of the present invention includes, but is not limited to, angiosperms and gymnosperms, such as potato, tomato, tobacco, avocado, alfalfa, lettuce, carrot, strawberry, sugarbeet, cassava, sweet potato, soybean, pea, bean, cucumber, grape, brassica, maize, turf grass, wheat, rice, barley, sorghum, oat, oak, eucalyptus, walnut, and palm. Thus, a plant may be a monocot or a dicot. “Plant” and “plant material,” also encompasses plant cells, seed, plant progeny, propagule whether generated sexually or asexually, and descendents of any of these, such as cuttings or seed. “Plant material” may refer to plant cells, cell suspension cultures, callus, embryos, meristematic regions, callus tissue, leaves, roots, shoots, gametophytes, sporophytes, pollen, seeds, germinating seedlings, and microspores. Plants may be at various stages of maturity and may be grown in liquid or solid culture, or in soil or suitable media in pots, greenhouses or fields. Expression of an introduced leader, trailer or gene sequences in plants may be transient or permanent.

In addition to plant-derived elements, such elements can also be identified in, for instance, fungi and mammals. Sequences from several species have already been shown to be accessible to Agrobacterium-mediated transformation. See Kunik et al., Proc Natl Acad Sci USA 98: 1871-1876 (2001), and Casas-Flores et al., Methods Mol Biol 267: 315-325 (2004), which are incorporated herein by reference.

### Promoters

The polynucleotides of the present invention can be used for specifically directing the expression of polypeptides or proteins in the tissues of plants. The nucleic acids of the present invention can also be used for specifically directing the expression of antisense RNA, or RNA involved in RNA interference (RNAi) such as small interfering RNA (siRNA), in the tissues of plants, which can be useful for inhibiting or completely blocking the expression of targeted genes. As used herein, “coding product” is intended to mean the ultimate product of the nucleic acid that is operably linked to the promoters. For example, a protein or polypeptide is a coding product, as well as antisense RNA or siRNA which is the ultimate product of the nucleic acid coding for the antisense RNA. The coding product may also be non-translated mRNA. The terms polypeptide and protein are used interchangeably herein. As used herein, promoter is intended to mean a nucleic acid, preferably DNA that binds RNA polymerase and/or other transcription regulatory elements. As with any promoter, the promoters of the current invention will facilitate or control the transcription of DNA or RNA to generate an mRNA molecule from a nucleic acid molecule that is operably linked to the promoter. The RNA may code for a protein or polypeptide or may code for an RNA interfering, or antisense molecule. As used herein, “operably linked” is meant to refer to the chemical fusion, ligation, or synthesis of DNA such that a promoter-nucleic acid sequence combination is formed in a proper orientation for the nucleic acid sequence to be transcribed into an RNA segment. The promoters of the current invention may also contain some or all of the 5′ untranslated region (5′ UTR) of the resulting mRNA transcript. On the other hand, the promoters of the current invention do not necessarily need to possess any of the 5′ UTR.

A promoter, as used herein, may also include regulatory elements. Conversely, a regulatory element may also be separate from a promoter. Regulatory elements confer a number of important characteristics upon a promoter region. Some elements bind transcription factors that enhance the rate of transcription of the operably linked nucleic acid. Other elements bind repressors that inhibit transcription activity. The effect of transcription factors on promoter activity may determine whether the promoter activity is high or low, i.e. whether the promoter is “strong” or “weak.”

In another embodiment, a constitutive promoter may be used for expressing the inventive polynucleotide sequences.

In another embodiment, a variety of inducible plant gene promoters can be used for expressing the inventive polynucleotide sequences. Inducible promoters regulate gene expression in response to environmental, hormonal, or chemical signals. Examples of hormone inducible promoters include auxin-inducible promoters (Baumann et al. Plant Cell 11:323-334 (1999)), cytokinin-inducible promoter (Guevara-Garcia Plant Mol. Biol. 38:743-753 (1998)), and gibberellin-responsive promoters (Shi et al. Plant Mol. Biol. 38:1053-1060 (1998)). Additionally, promoters responsive to cold temperature, drought, salt stress, or Abscisic Acid may be used for expressing the inventive polynucleotide sequences. SEQ ID NO: 1 is illustrative of such a promoter.

### Nucleic Acid Constructs

The present invention provides constructs comprising the isolated nucleic acid molecules and polypeptide sequences of the present invention. In one embodiment, the DNA constructs of the present invention are Ti-plasmids derived from A. tumefaciens.

In developing the nucleic acid constructs of this invention, the various components of the construct or fragments thereof will normally be inserted into a convenient cloning vector, e.g., a plasmid that is capable of replication in a bacterial host, e.g., E. coli. Numerous vectors exist that have been described in the literature, many of which are commercially available. After each cloning, the cloning vector with the desired insert may be isolated and subjected to further manipulation, such as restriction digestion, insertion of new fragments or nucleotides, ligation, deletion, mutation, resection, etc. to tailor the components of the desired sequence. Once the construct has been completed, it may then be transferred to an appropriate vector for further manipulation in accordance with the manner of transformation of the host cell.

A recombinant DNA molecule of the invention typically includes a selectable marker so that transformed cells can be easily identified and selected from non-transformed cells. Examples of such markers include, but are not limited to, a neomycin phosphotransferase (nptII) gene (Potrykus et al., Mol. Gen. Genet. 199:183-188 (1985)), which confers kanamycin resistance. Cells expressing the nptII gene can be selected using an appropriate antibiotic such as kanamycin or G418. Other commonly used selectable markers include the bar gene, which confers bialaphos resistance; a mutant EPSP synthase gene (Hinchee et al., Bio/Technology 6:915-922 (1988)), which confers glyphosate resistance; and a mutant acetolactate synthase gene (ALS), which confers imidazolinone or sulphonylurea resistance (European Patent Application 154,204, 1985).

Additionally, vectors may include an origin of replication (replicons) for a particular host cell. Various prokaryotic replicons are known to those skilled in the art, and function to direct autonomous replication and maintenance of a recombinant molecule in a prokaryotic host cell.

The vectors will preferably contain selectable markers for selection in plant cells. Numerous selectable markers for use in selecting transfected plant cells including, but not limited to, kanamycin, glyphosate resistance genes, and tetracycline or ampicillin resistance for culturing in E. coli, A. tumefaciens and other bacteria.

For secretion of the translated protein into the lumen of the endoplasmic reticulum, the periplasmic space or into the extracellular environment, appropriate secretion signals may be incorporated into the expressed polypeptide. The signals may be endogenous to the polypeptide or they may be heterologous signals.

In one embodiment, a DNA construct of the current invention is designed in a manner such that a polynucleotide sequence described herein is operably linked to a tissue-specific promoter.

In a further embodiment, the DNA constructs of the current invention are designed such that the polynucleotide sequences of the current invention are operably linked to DNA or RNA that encodes antisense RNA or interfering RNA, which corresponds to genes that code for polypeptides of interest, resulting in a decreased expression of targeted gene products. The use of RNAi inhibition of gene expression is described in U.S. Pat. No. 6,506,559, and the use of RNAi to inhibit gene expression in plants is specifically described in WO 99/61631, both of which are herein incorporated by reference.

The use of antisense technology to reduce or inhibit the expression of specific plant genes has been described, for example in European Patent Publication No. 271988. Reduction of gene expression led to a change in the phenotype of the plant, either at the level of gross visible phenotypic difference, for example a lack of lycopene synthesis in the fruit of tomato leading to the production of yellow rather than red fruit, or at a more subtle biochemical level, for example, a change in the amount of polygalacturonase and reduction in depolymerisation of pectins during tomato fruit ripening (Smith et. al., Nature, 334:724-726 (1988); Smith et. al., Plant Mol. Biol., 14:369-379 (1990)). Thus, antisense RNA has been demonstrated to be useful in achieving reduction of gene expression in plants.

In one embodiment an inventive polynucleotide sequence is capable of being transcribed inside a plant to yield an antisense RNA transcript is introduced into the plant, e.g., into a plant cell. The inventive polynucleotide can be prepared, for example, by reversing the orientation of a gene sequence with respect to its promoter. Transcription of the exogenous DNA in the plant cell generates an intracellular RNA transcript that is “antisense” with respect to that gene.

The invention also provides host cells which comprise the DNA constructs of the current invention. As used herein, a host cell refers to the cell in which the coding product is ultimately expressed. Accordingly, a host cell can be an individual cell, a cell culture or cells as part of an organism. The host cell can also be a portion of an embryo, endosperm, sperm or egg cell, or a fertilized egg.

Accordingly, the present invention also provides plants or plant cells, comprising the DNA constructs of the current invention. Preferably the plants are angiosperms or gymnosperms. The expression construct of the present invention may be used to transform a variety of plants, both monocotyledonous (e.g. wheat, turf grass, maize, rice, oat, wheat, barley, sorghum, orchid, iris, lily, onion, banana, sugarcane, and palm), dicotyledonous (e.g., Arabidopsis, potato, tobacco, tomato, avocado, pepper, sugarbeet, broccoli, cassava, sweet potato, cotton, poinsettia, legumes, alfalfa, soybean, pea, bean, cucumber, grape, brassica, carrot, strawberry, lettuce, oak, maple, walnut, rose, mint, squash, daisy, and cactus, oaks, eucalyptus, maple), and Gymnosperms (e.g., Scots pine; see Aronen, Finnish Forest Res. Papers, Vol. 595, 1996), white spruce (Ellis et al., Biotechnology 11:84-89, 1993), and larch (Huang et al., In Vitro Cell 27:201-207, 1991).

### Plant Transformation and Regeneration

The present polynucleotides and polypeptides may be introduced into a host plant cell by standard procedures known in the art for introducing recombinant sequences into a target host cell. Such procedures include, but are not limited to, transfection, infection, transformation, natural uptake, electroporation, biolistics, and Agrobacterium. Methods for introducing foreign genes into plants are known in the art and can be used to insert a construct of the invention into a plant host, including, biological and physical plant transformation protocols. See, for example, Miki et al., 1993, “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Horsch et al., Science 227:1229-31 (1985)), electroporation, micro-injection, and biolistic bombardment.

Accordingly, the present invention also provides plants or plant cells, comprising the polynucleotides or polypeptides of the current invention. In one embodiment, the plants are angiosperms or gymnosperms. Beyond the ordinary meaning of plant, the term “plants” is also intended to mean the fruit, seeds, flower, strobilus etc. of the plant. The plant of the current invention may be a direct transfectant, meaning that the vector was introduced directly into the plant, such as through Agrobacterium, or the plant may be the progeny of a transfected plant. The progeny may also be obtained by asexual reproduction of a transfected plant. The second or subsequent generation plant may or may not be produced by sexual reproduction, i.e., fertilization. Furthermore, the plant can be a gametophyte (haploid stage) or a sporophyte (diploid stage).

In this regard, the present invention contemplates transforming a plant with one or more transformation elements that genetically originate from a plant. On the other hand, the plant that is to be transformed, may be transformed with a transformation cassette that contains one or more genetic elements and sequences that originate from a plant of a different species. It may be desirable to use, for instance, a cleavage site, that is native to a potato genome in a transformation cassette or plasmid for transforming an alfalfa plant.

Specific examples are presented below of methods for obtaining an inducible promoter, as well as for introducing an expression cassette, via Agrobacterium, to produce transgenic plants. They are meant to be exemplary and not as limitations on the present invention.

### Example 1

### Selection of Candidate Regulatory Elements from Alfalfa

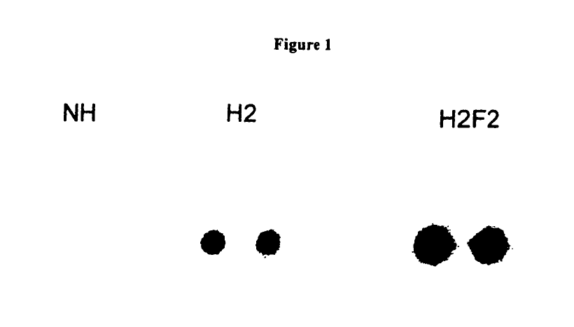

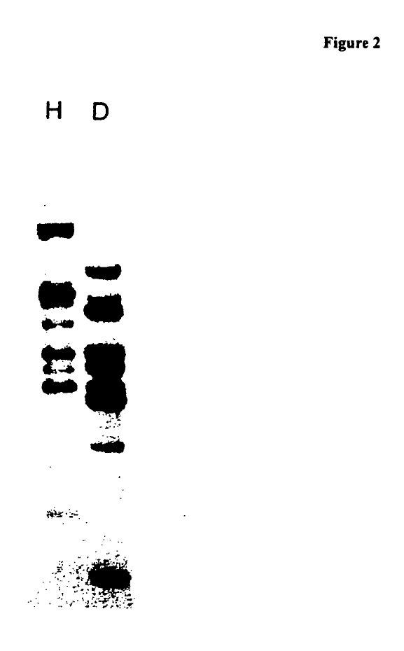

An expression profile was generated following macroarray hybridizations, as described below in Example 2, using cDNA probes isolated from alfalfa leaves mRNA that were acclimated to the following conditions: non-hardened (NH), hardened two weeks at 2° C. (H2), and hardened two weeks at 2° C. followed by two weeks at −2° C. (H2F2). One clone was chosen for its high level of expression at low temperature and its very low expression at room temperature (FIG. 1). This clone codes for a dehydrin homolog. Moreover, the allele content of this alfalfa gene has been verified by Southern hybridization (FIG. 2). Because a dehydrin homolog has been identified, it is possible to identify and characterize the promoter and terminator sequences responsible for low temperature induction.

### Example 2

### Macroarray Analysis

Double-stranded cDNA were synthesized from mRNA using the Universal RiboClone cDNA Synthesis System (Promega, San Luis Obispo, Calif.) according to the manufacturer instructions. mRNA was probed with oligo-(dT) 15 primer. Following the second strand synthesis, a ligation using E. coli DNA ligase was performed. Twenty-five ng of double-stranded cDNA was radioactively labeled with alpha 33P-dCTP. Probes were purified using mini Quick Spin DNA Columns (Roche Applied Science, Laval, Quebec). Membranes were hybridized overnight at 68° C. as previously described. Detection was carried out using a phosphorimager BAS-1000 (Fudji, Tokyo, Japan) or a Storm (Amersham Bioscience, Baie d'Urfe, Canada). Data were analysed using ArrayGauge v1.2 (Fudji, Tokyo, Japan).

### Example 3

### Analysis of Cold-Inducibility in Alfalfa Leaves

### A. Plant Material

Alfalfa plants were grown in tubes or pots filled with soil under the following environmentally controlled conditions: 21° C./17° C. (day/night) temperature, 16 h photoperiod, and 250 μmol m−2 s−1 photsynthetic photon flux density provided by a mixture of Cool White (VHO) fluorescent (GTE, Sylvania) and incandescent lamps. Plants were watered daily and fertilized once a week with 1 g/liter solution of a commercial fertilizer (20-20-20, Plant Prod, Brampton, Ontario, Canada) containing micronutrients.

### B. Hardening Treatments

Five weeks after planting, environmental condition were changed to a constant temperature of 2° C., 8 h photoperiod, and 150 μmol m⁻² s⁻¹ photosynthetic photon flux density. Plants were allowed to acclimate for two weeks under these conditions. After this initial acclimation period, a group of plants was transferred to a freezer at −2° C. in the dark for an additional two week period. Thermocouples were inserted in pots to insure that free water in soil and plants was frozen and that equilibrium was achieved (Gullord, M, et al. Crop. Sci. 15: 153-157 (1975).

### C. RNA Extraction and Dot-Blot Analysis

Total RNA was extracted according to standard procedures (De Vries, S., Hoge, H. and Bisseleing, T. (1988) Isolation of total and polysomal RNA from plant tisssues. Plant Molecular Biology manual B6: 1-6). Samples varying from 0.5 to 1.0 g were ground to a fine powder in liquid N2 using a mortar and pestle. Samples were kept frozen at −80° C. until extraction in 2 ml per g fresh weight of a preheated (90° C.) 1:1 mixture of RNA extraction buffer (100 mM Tris-NaOH (pH 9.0), 1% w/v SDS, 100 mM LiCl, 10 mM EDTA) and phenol containing 0.1% (w/v) 8-hydroxy quinoline that was previously equilibrated with TLE (200 mM Tris-HCl (pH 8.0), 100 mM LiCl, 5 mM EDTA). Tubes were placed on a rotary shaker at 300 rpm at room temperature for 5 min. Chloroform was added (1 ml per g fresh weight) to the homogenate and shaking was continued for 15 min at room temperature. Extracts were centrifuged at 2500 rpm on a clinical IEC HN-SII centrifuge (International Equipment Company, Massachusetts) for 10 min at room temperature. After centrifugation, the aqueous phase was removed and reextracted with chloroform and centrifuged at 12000×g for 10 min. The volume of the aqueous phase obtained after the second centrifugation was measured and total RNA was precipitated with 2M LiCl at 4° C. for 16 h. Tubes were centrifuged at 12000×g for 30 min at 4° C. The pellet was washed with 2M LiCl and twice with 80% (v/v) ethanol. The vacuum dried pellet was dissolved in TE (10 mM Tris-HCl (pH 7.4), 1 mM EDTA) and stored at −80° C. Total RNA was quantified by UV absorption at 260 nm and integrity was verify by size distribution analysis (relative to RNA mol wt markers, BRL) on denaturing agarose formaldehyde gels. Fourney, R. M., et al. Focus 10: 5-7 (1988).

Five micrograms of total leaf RNA was applied to a nylon membrane (Immobilon NY+, Millipore, Canada). DNA probes were radioactively labeled (Rediprime™ II Random Prime Labelling System, Amersham Bioscience, Canada) with alpha-³²P-dCTP and hybridization were done at 68° C. using 2×SSC containing 0.5% (w/v) sodium dodecyl sulfate (SDS) and 0.25% (w/v) low fat milk powder. GeHealthcare Hyperfilm™ was used for autoradiography. FIG. 1 presents a dot blot analysis of dehydrin inducibility in alfalfa leaves.

### Example 4

### DNA Extraction and Southern Analysis

DNA was extracted by the cetyltrimethylammonium bromide (CTAB) method as described by Rogers and Bendich (Rogers, S. O. and Bendich, A. (1988) Extraction of DNA from plant tissues. Plant Molecular Biology manual A6: 1-10.) with modifications to optimize extraction for alfalfa. Samples of 2 g were ground to a fine powder in liquid N2 using mortar and pestle. Samples were kept at −80° C. until extraction with 1.5 ml per g fresh weight of a preheated (65° C.) 1×CTAB buffer [1% (w/v) cetyltrimethylammonium bromide, 100 mM Tris-HCl (pH 8.0), 20 mM EDTA (pH 8.0), 1.4M NaCl, 1% polyvinylpyrrolidone Mr 40000]. Tubes were maintained in hot water (65° C.) for 5 min. One volume of chloroform:isoamyl alcohol (24:1) was added to each tube, mix and centrifuge for 4 min at 2500 rpm with the clinical centrifuge IEC HN-SII. After centrifugation, the aqueous phase was removed and reextracted with one volume of chloroform:isoamyl alcohol and centrifuged at 12000×g for 5 min. To the aqueous phase, 1/10 volume of CTAB 10% (10% CTAB, 0.7M NaCl) was added and mixed gently. Another extraction with one volume of chloroform:isoamyl alcohol was performed and the supernatant was precipitated by adding one volume of precipitating buffer [1% CTAB, 50 mM Tris-HCl (pH 8.0), 10 mM EDTA (pH 8.0)]. The tubes were mixed and centrifuged for 2 min. The supernatant was discarded and 150 μl of high salt TE buffer (10 mM Tris-HCl (pH 8.0), 1 mM EDTA (pH 8.0), 1M NaCl) was added. Each tube was heated at 65° C. to assist DNA rehydration. DNA was precipitated by adding two volumes of cold 95% ethanol for 16 h. After centrifugation, the pellet was washed with 80% (v/v) ethanol, vacuum dried, and dissolved in 0.1×TE buffer. Each samples of DNA was treated with a RNase, DNase free from bovine pancreas (Roche Diagnostics, Canada).

Approximately 12 μg of total genomic DNA was digested with the restriction endonucleases HindIII and DraI, electrophoresed on a 0.8% agarose gel, and transferred onto a nylon membrane (Hybond-N, GeHealthcare). Hybridization procedures were performed using a 32P-labeled dehydrin cDNA according to the methods described by Sambrook et al. (Sambrook, J., Fritsch, E. F. and Maniatis, T. (1989) Molecular cloning: a laboratory manual, 2^{nd} ed. Cold Spring Harbor Laboratory Press. Cold Spring Harbor, N.Y.). FIG. 2 shows the autoradiograph of Southern analysis of the dehydrin gene(s) in alfalfa.

### Example 5

### Isolation of the Dehydrin Promoter and Terminator by Genome Walking

The 5′-flanking region of the dehydrin gene was isolated according to the protocol of the Universal Genome Walker Kit (Clontech, Palo Alto, Calif.). Genomic DNA was extracted from leaves of Medicago sativa genotype 11.9, and was further digested with five blunt-cutting restrictions enzymes: DraI, EcoRV, PvuII and StuI. Each batch of digested DNA fragments was then ligated to a genome walker adaptor to generate five Genomic Walker libraries. Two successive rounds of PCR reactions were carried out on each genome walker library to obtain the promoter fragment of the dehydrin gene. The primary PCR was performed with the gene specific primer GSP1 (5′-CCATATTCATCAACCCTACCAGTTTG-3′) and the adaptor primer from the kit (AP1) in the GeneAmp PCR System 9600 (Perkin-Elmer 9600 turbo). The resulting PCR products were diluted and used as templates for the secondary PCR reaction, using the nested specific primer GSP2 (5′-CCACCTTGTTGATATTGAGACATTG-3′) and the nested adaptor primer (AP2). Amplification conditions for the first PCR was as following: 7 cycles of 94° C. for 2 s and 68° C. for 3 min after an initial denaturation step at 94° C. for 3 min, followed by 32 cycles of 94° C. for 2 s and 63° C. for 3 min, and a final extension at 63° C. for 4 min. The same conditions were used for the second round of PCR except that the number of denaturation and annealing/extension cycles was reduced to 5 cycles followed by another 20 cycles. The analysis of the nested PCR reactions of the EcoRV library by agarose gel electrophoresis revealed several bands. An amplified 1.5 kb fragment was cloned into pGEM-T Easy vector (Promega, Madison, Wis.) and then sequenced to confirm the promoter region of the dehydrin gene. The dehydrin promoter sequence is set forth in SEQ ID NO: 1.

In order to obtain the 3′ region of the dehydrin gene, the downstream walking was performed similar to the upstream walking, using the adaptor primers (AP1, AP2) and the following DH specific primers: DH1-3′ (5′-GACATGATGAGCAAC ACCTTGGTGAGA-3) instead of GSP1 and DH2-3′ (5′-GGATAAGATTAAGGAGAA GCTTCCCGGT-3) instead of GSP2. The temperatures for annealing and extension for the first and second PCR steps were 70° C./65° C. and 72° C./67° C. respectively. Other PCR conditions, including cycle number, were the same as described previously for isolating the dehydrin promoter. The second PCR reaction from the PvuII library generated a 4 kb fragment. The fragment was cloned into plasmid pGEMT-easy vector for sequencing. The dehydrins terminator is set forth in SEQ ID NO: 2.

### Example 6

### DNA Sequencing

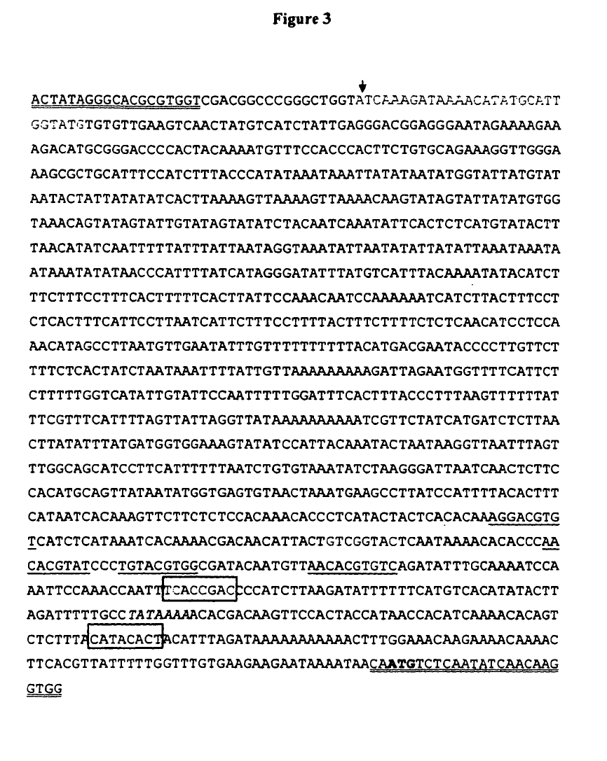

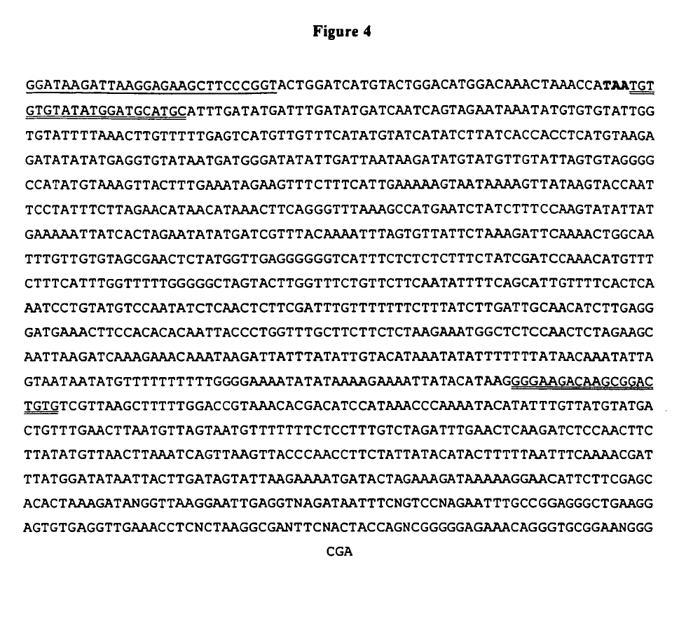

DNA sequencing was performed as described by Sanger, F. et al. Proceedings of the National Academy of Sciences, USA 74, 5463-5467 (1977). Sequence analysis was performed to confirm the identity of the fragments amplified by genome walking. FIG. 3 presents the sequence of the fragment obtained from the amplification upstream of the dehydrin cDNA (also set forth in SEQ ID NO: 1). FIG. 4 presents the sequence of the fragment obtained from the amplification downstream of the dehydrin cDNA (also set forth in SEQ ID NO: 2).

Several cis-acting regulatory elements were identified in the promoter region of alfalfa dehydrin (see FIG. 3 for localization). The core sequence CCGAC LTRE has been reported to be important for cold responsive gene expression of the Arabidopsis gene COR15A (Baker et al. Plant Mol. Biol. 24: 701-713 (1994)), the Brassica napus BN115 (Jiang C et al. Plant Mol. Biol. 30: 679-684 (1996)) and the wheat dehydrin gene WCS120 (Ouellet F. et al. Febs Lett. 423: 324-328 (1998)). The dehydrin promoter also contains four sequences with significant homologies with ABRE-like motifs. These elements contain a core sequence ACGT that have been previously identified in the G-box like motif. They are sufficient to confer ABA responsive gene expression.

### Example 7

### Construction of Expression Cassettes

The cassette for expression analysis using the GUS reporter gene was assembled as follows. A promoterless GUS gene fused to the NOS terminator was digested from pBI101 with HindIII and EcoRI, and was inserted into the corresponding sites of the pUC19 polycloning site. The resulting plasmid was named pBI201 and was used as intermediate vector for the first construct described here (pR720).

The strategy used for linking the fragment corresponding to the dehydrin promoter with the GUS coding region involves three PCR reactions and was described by Darveau, A. et al. Methods in Neurosciences 26: 77-85 (1995). In the first reaction, two primers flanking the dehydrin upstream sequence, AP2 and 3B-DEHYDRIN-GUS.R2 (5′-GTTTCTACAGGACGTAACATTGTTATTTTATTCTTCTTCACAAACC-3′), were used to amplify the promoter. The underlined portion of the lower gene specific primer 3B-DEHYDRIN-GUS.R2 anneals with the first 20 pb of the GUS gene coding sequence. In the second PCR reaction, 228 pb of the GUS coding region were recovered using the primer pair GUS1.C (5′-ATGTTACGTCCTGTAGAAACC-3′) and GUS228.R (5′-TCGGTATAAAGACTTCGCGCTGAT-3′).

Products of these two PCR steps were mixed and subjected to a third amplification using AP2 and GUS228.R as upper and lower primers, respectively. PCR fragment generated was digested with XmaI and MfeI, and then inserted in pBI201 previously digested with the same restriction enzymes. A HindIII-EcoRI fragment carrying the promoter region of alfalfa dehydrin, the GUS reporter gene and the nopaline synthase transcriptional terminator (NOS) was taken from the resulting plasmid and subcloned into pCambia2300 (Cambia, Canberra, Australia) to assemble the plant expression vector pR720.

For the construction of the cassette containing the dehydrin terminator downstream of the GUS gene in addition to the dehydrin promoter, the following primers were first used for amplification of the terminator from pGEM-T plasmid containing the terminator amplified by genome walking:

Dehydrin-18F-term-SacI-f:
(5′-TGCAGAGCTCTGTGTGTATATGGATGC-3′)
Dehydrin-18F-term-EcoRI-r:
(5′-AGCTGAATTCCACAGTCCGCTTGTCTTCCC-3′).

The primers have additional nucleotides at the 5′ end to produce suitable restrictions sites for cloning the PCR product (underlined portions). The amplified fragment containing recognition sites for EcoRI and SacI at its termini was isolated, digested, and cloned into the EcoRI and SacI restriction sites of plamid pR720. The generated plasmid was designated by pR705.

Two plant expression vectors were constructed in order to replace the GUS reporter gene in pR705 with coding regions of the heavy chain (HC) and light chain (LC) of the C5-1 monoclonal antibody. This was performed as described above for linking GUS to the dehydrin promoter. Darveau, A., et al. Methods in Neurosciences 26: 77-85 (1995).

In order to amplify a 380 pb localized at the 3′ end of the dehydrin promoter, the first PCR reaction was performed using primers PromDehyd.987C and PromDehyd.987C.

PromDehyd.987C
(5′-CTTCTCTCCACAAACACCCTC-3′)
HC-C5-1Dehyd.R
(5′-GCAAGGTCCACACCCAAGCCATTGTTATTTTATTCTTCTTCA
C-3′).

While several C5-1 monoclonal antibody sequences are publicly available through the National Center for Biotechnology Information (NCBI) GenBank®, the present invention follows the method for detecting C5-1 mAb in alfalfa as disclosed in Khoudi, H., et al. Biotechnol. Bioeng. 64 (2):135-43 (1999).

The primer pair HC-C5-1.C (5′-ATGGCTTGGGTGTGGACCTTGC-3′) and HC-C5-1-SACI.R (5′-ATAAGAGCTCTCATTTACCAGGAGAGTGGG-3′) was used for PCR amplification of coding region of HC-C5-1. Fragments resulting from these two PCR were used as template in the third PCR reaction using PromDehyd.987C and HC-C5-1-SACI.R as upper and lower primers. Fragment issued from this last PCR was digested by HpaI and SacI and further cloned into pR720 vector previously digested by the same enzymes. The generated plasmid was named pR725.

The second expression cassette harboring the LC-C5-1 under the control of alfalfa dehydrin promoter was constructed by using these following primers:

PromDehyd.987C,
LC-C5-1Dehyd.R
(5′CCAAGTATCTGAGGTGTGAAAACCATTGTTATTTTATTCTTCTTC
AC3′),
LC-C5-1.C
(5′-ATGGTTTTCACACCTCAGATACTTGG-3′)
and
LC-C5-1-SACI.R
(5′-ATATGAGCTCCTAACACTCATTCCTGTTGAAGC-3′).

LC-C5-1.C and LC-C5-1-SACI.R were positioned at the 5′ and 3′ end of the coding sequence of the light chain. The product of the third PCR reaction was digest by PmII and Sad and introduced into pR720. The resulting plasmid was named pR722. Digestion with restriction enzymes proved that all recombinant vectors had the inserts with expected length of the target fragment.

A construct expressing both the C5-1 antibody H and L chains was generated and named pR728. A HindIII-EcoRI fragment, blunted on the EcoRI side, which carries the C5-1 heavy chain flanked by dehydrin promoter and terminator, was excised from pR725 and inserted in pR722 previously cut with HindIII and SmaI.

### PCR Conditions

The cycling program used was as follows: an initial denaturation step of 5 min at 94° C., followed by 40 cycles of 1 min at 94° C., 1 min at 55° C. and 1 min at 72° C. A final extension period of 7 min at 72° C. was included in this program. Tgradient thermocycler (Biometra Wathman) was used for all PCR reactions.

### Protein Extraction

Proteins were extracted from frozen (−80° C.) leaves using extraction buffer (50 mM Tris-HCl, pH 7.4, 150 mM NaCl and 0.1% (v/v) TritonX-100) supplemented with 10 μM chymostatine and 1 mM PMSF. The soluble fraction was recovered by centrifugation at 20 000 g for 20 min, and the concentration of proteins was determined according to the method of Bradford (1976, Anal. Biochem. 72: 248) using bovine serum albumin as a standard.

### Example 8

### Elisa Assays and Western Blot Analysis

The level of C5-1 in alfalfa infiltrated leaves was determined both by Elisa and Western blot. For the Elisa assay, the wells of microplates (Immulon 2HB, Thermo. LabSystems) were coated overnight with goat anti-mouse IgG diluted at 2 μg/mL in a sodium carbonate buffer (50 mM, pH 9.6). PBS containing 0.25% casein (PBS-casein) and 0.05% tween-20 was used as blocking agent. After dilution in PBS-casein, protein extracts were applied to the Elisa coated plates and incubated for 1 h at 37° C. The plates were washed and the binding of C5-1 was revealed using a peroxidase-conjugated goat anti-mouse IgG (Jackson Imm. Res. Lab). The revelation was performed with the Immunopure TMB (Pierce) substrate, and optical density at 450 nm was measured on a microplate reader (Thermo. LabSystems). Antibody concentrations (ng/ml) in leaf extracts were calculated from a standard curve generated with murine serum (ICN Pharmaceuticals).

For immunoblot analysis, equal amounts of soluble proteins (50 μg) were separated by SDS-PAGE on a 10% gel under non-reducing conditions. The separated polypeptides were transferred electrophoretically for 1 h to a PVDF membrane (Roche Diagnostics) using a Western transfer apparatus (Bio-Rad). After transfer, the membrane was blocked overnight at 4° C. with 5% (w/v) dry milk in Tris-Buffered saline (TBS) containing 0.1% Tween-20. The membrane was then probed with a peroxidase conjugated goat anti-mouse IgG (H+L) (Jackson Imm. Res. Lab) at a 1:10000 dilution (1 h in a blocking solution containing 2% of milk powder). After washing with TBS-Tween 20, antibody binding was detected by chemiluminescence using the Boehringer Manheim BM chemiluminescence kit (Roche Biochemicals, Laval, Canada). The blots were exposed to Kodak Biomax MS films (Eastman Kodak Rochester, N.Y.) for 5 min. To confirm equal sample loading, membranes were stained with Ponceau red before the antibody reaction. Infiltrated leaves with empty pCambia2300 vector were used as negative control. Purified C5-1 was mixed with total soluble proteins from control infiltrated alfalfa leaves and loaded as standard.

### Example 9

### Agroinfiltration

Expression of C5-1 Abs was shown using a transient-expression system employing alfalfa leaves and Agrobacterium activated with acetosyringone. Agroinfiltration of alfalfa leaves (infiltration of Agrobacterium) is performed essentially as described by Kapila et al. Plant Science 122: 101-108 (1997). Alfalfa seeds of the cultivar Oneida VR are subject to mechanical scarification for improving germination rates. Germination was carried out at 20° C. under a 16 h light/8 hours dark cycle. Plants were covered by a seed tray incubator to maximize humidity, and allowed to develop for 2 weeks before the leaves were collected for agroinfiltration.

Cultures of Agrobacterium (AGL1 strain), containing the binary plasmid of interest, were grown at 28° C. overnight in YEB (5 g/l beef extract, 1 g/l yeast extract, 5 g/l peptone, 5 g/l sucrose, 2 mM MgSO₄) supplemented with 10 mM 2-(N-morpholino)ethanesulfonic acid (MES), and adjusted to pH 5.6. Acetosyringone (20 μM) and appropriate antibiotics (kanamycin: 50 mg/L, carbenicillin: 25 mg/L) were added to the culture medium. Bacteria were grown to OD 600 nm of 0.8 and then centrifuged at 10000 g for 8 min. Bacterial cells were resuspended in ½ MMA medium (½ MS micro- and macronutrients, 5 mM 2-(N-morpholino)ethanesulfonic acid (MES), adjusted to pH 5.6, and supplemented with sucrose (60 g/L) and acetosyringone (200 μM) to an OD600 of 2.4. Cells were kept at room temperature for 1 h before infiltration.

The collected alfalfa leaves were placed in the Agrobacterium suspension and the mixture was placed for 30 min in a vacuum chamber under >99% vacuum with gentle agitation. The infiltrated leaves were laid on a Whatman filter paper (previously wetted in 1.5 mL B5H salts supplemented with 200 μM acetosyringone) inside a petri dish. As a control, leaves were incubated in a Petri dish for 4 days at 23° C. before expression analysis was performed. For cold induction, leaves were incubated for 3 days at 23° C. and then transferred for 14, 7, or 4 days at 2° C. Leaves were homogenized and the homogenate was assayed for assembled C5-1 mAbs using the Elisa assay as described above.

In these experiments, an equal mixture of two populations of Agrobacterium containing either the heavy or light chain constructs was used for infiltration studies. Most of the infected cells were occupied by both strains, leading to accumulation of functional and fully assembled C5-1 in the infiltrated leaf extracts. Expression of C5-1 antibody was performed under the control of both the alfalfa dehydrin and the dual enhanced Cauliflower mosaic virus 35S (2×35S/TEV) promoters. 2×35S/TEV promoter consists of the core 35S promoter with a dual enhancer fused to the TEV translational enhancing leader sequence from the tobacco etch virus.

The expression of C5-1 under the control of the dehydrin promoter was strongly induced by low temperatures. When induced, the DH-C5-1-DH constructs (722+725) resulted in more than 15 fold the level of C5-1 expression obtained in unstressed leaves. Comparison of the relative strength of tested constructs shows that the accumulation of C5-1 antibodies was 1.7× (on average) greater when expression was driven by the dehydrin promoter than that observed with the 2×35STEVpromoter (512+513). Moreover, in leaves infiltrated with cassettes harboring the dehydrin promoter and terminator (722+725), C5-1 expression reached 0.095% of total soluble protein, where as the 2×35S/TEV constructs yielded a maximum level of 0.027% of total soluble protein. These results illustrate that the 5′-flanking sequence of the dehydrin gene has the capacity to drive high and cold inducible expression of C5-1 in alfalfa leaves.

The results indicate that cold acclimation leads to a strong accumulation of C5-1 antibody in the case of DH-C5-1-DH constructs (722+725). This is consistent with the ELISA results in Example 8. When C5-1 was expressed under the control of 2×35S/TEV promoter, the amount of C5-1 protein obtained in leaves was lower than that observed when using the dehydrin promoter. Thus, under stress conditions, the stress-inducible dehydrin promoter promotes an enhanced production of C5-1, compared to the constitutive expression driven by the 2×35S/TEV promoter.

In order to develop an efficient cold-inducible expression system for protein expression in plant, the induction time should be taken into consideration. To test whether the response of dehydrin promoter could require less than 14 days of cold treatment, C5-1 expression was measured after 7 days of cold treatment. In this experiment, R512+R513 co-infiltration was used as a positive control. The dehydrin promoter provides the highest expression levels of C5-1 antibody (0.03% TSP). In cold acclimated alfalfa leaves infiltrated with constructs carrying the dehydrin promoter and terminator (722+725), the amount of C5-1 Abs increased 9 times by cold treatment. Leaves carrying the DH-C5-1-DH constructs still exhibit an enhanced level of C5-1 antibody after only 7 days of cold treatment, indicating that the activity of the alfalfa dehydrin promoter was sustained for several days under low temperature conditions. We previously demonstrated that mRNA accumulation of the endogenous dehydrin gene reached a maximum after only 4 days of cold treatment of detached alfalfa leaves (data not shown).

A drastic increase in the expression level of C5-1 mAbs in agro-infiltrated leaves was observed after only 4 days of exposure to low temperatures. Cold treatment induced a 13-fold increase in C5-1 accumulation over control leaves. These results clearly indicate that the expression cassette carrying both the heavy and the light chain under the control of the alfalfa dehydrin promoter was able to mediate the induction of C5-1 mAbs expression under cold treatment.

### Example 10

### Agrobacterium-Mediated Alfalfa Transformation

Alfalfa genotype R2336 was used for transformation. Alfalfa young petioles (2 cm) were cut from in vitro maintained plants and were incubated on solid modified S2 KH medium (with 25 mM potassium sulphate, 2.5 mM praline, 45.8 mg/L Fe(III) EDTA and 3% sucrose) for two days in the growth culture (25° C. under 25 μE/m2 sec of light in a 16 hours light/8 hours dark cycle). Agrobacterium tumefaciens strains Agl1 was used for stable transformation. The Agrobacterium clones containing a binary vector previously described in Example 9 were grown for 24 hours at 28° C. in 2 mL of LB medium containing 25 μg/mL carbenicilin and 50 μg/mL kanamycin, respectively. On the transformation day, the petioles were co-cultivated with bacteria for 2 min, blotted on sterile filter paper, and cultured on antibiotics free SH2K medium supplemented with 20 μM acetosyringone. Explants were incubated for two days in the culture chamber. After the co-cultivation period, explants are cut in two pieces, transferred to SH2K media supplemented with 300 mg/L timentin and 50-75 mg/L kanamycin (for R2336 and N442 genotypes respectively), and placed back in the culture chamber for 6 weeks. The explants are transferred onto fresh medium every two weeks.

For development of embryos, callus are transferred onto Boi2Y medium supplemented with 300 mg/L timentin and 50-75 mg/L kanamycin (for 82336 and N442 genotypes respectively), and replaced in the culture chamber for two to eight weeks. The developing embryos are transferred on kanamycin 100 mg/L MS medium (Murashige, T. and Skoog, F. Physiologia Plantarum 15:473-497 (1962)) supplemented with 300 mg/L timentin and incubated for three to four additional weeks.

Embryos that have developed at least 1 root and 1 trifoliate leaflet were then transferred into sterile tubes containing a mix of vermiculite/perlite supplemented with half-MS media and are incubated for 10 days in a growth chamber (25° C., 50-60% humidity, 80 μE/m2 sec on light intensity, 16 h light/8 hours dark cycle). Then the plantlets were transported to the greenhouse for acclimation and growth.

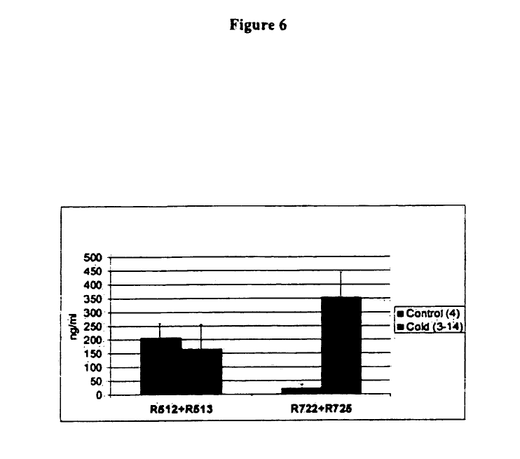

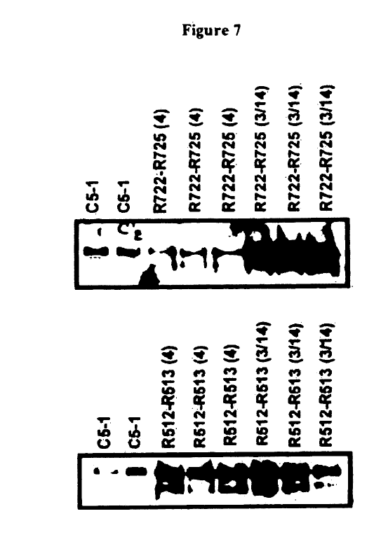

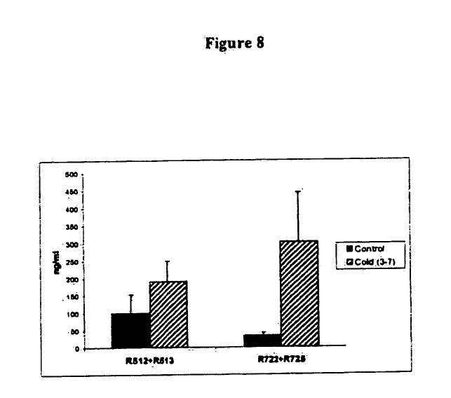

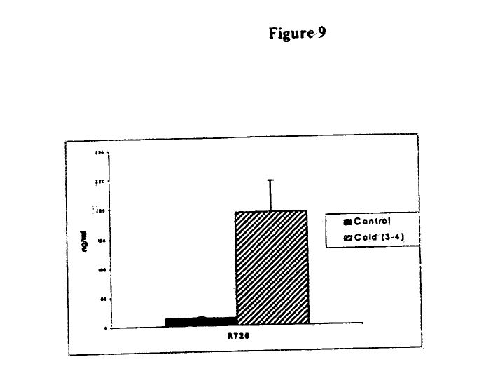

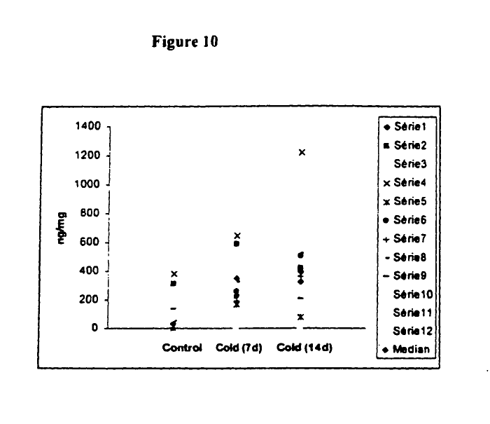

Based on the results shown in FIGS. 6-10, it is clear that the alfalfa Dehydrin promoter and terminator direct strong and cold inducible expression of C5-1. Plants exposed to cold during 7 days had 6-8 fold increase (based on median value) in C5-1, compared with untreated transgenic plants. Hardening prolonged to 14 days led to better accumulation of C5-1 in some transgenic plants.

What is claimed is:

1. An isolated nucleic acid sequence comprising a nucleotide sequence selected from the group consisting of SEQ ID NO: 1, 2 and a variant of SEQ ID NO: 1 or 2 that has a sequence identity that is greater than or equal to 60%.

2. An expression cassette comprising a promoter having a nucleotide sequence selected from the group consisting of SEQ ID NO: 1, 2 and a variant of SEQ ID NO: 1 or 2 that has a sequence identity that is greater than or equal to 60%.

3. The expression cassette of claim 2, wherein the promoter is operably linked to a nucleotide sequence coding for a protein.

4. A method of expressing a protein in a transformed plant cell comprising transforming said cell with the expression cassette of claim 3.

5. The method of claim 4 wherein the plant cell is an alfalfa cell.


SEQUENCE LISTING (20 sequences)

SEQ ID NO: 1 - DNA, 1384 bp, Artificial Sequence
  Description of Artificial Sequence Synthetic polynucleotide
atcaaagataaaacatatgcattggtatgtgtgttgaagtcaactatgtcatctattgagggacggagggaatagaaaagaaagacatgcgggaccccactacaaaatgtttccacccacttctgtgcagaaaggttgggaaagcgctgcatttccatctttacccatataaataaattatataatatggtattatgtataatactattatatatcacttaaaagttaaaagttaaaacaagtatagtattatatgtggtaaacagtatagtattgtatagtatatctacaatcaaatattcactctcatgtatactttaacatatcaatttttatttattaataggtaaatattaatatattatattaaataaataataaatatataacccattttatcatagggatatttatgtcatttacaaaatatacatctttctttcctttcactttttcacttattccaaacaatccaaaaaatcatcttactttcctctcactttcattccttaatcattctttccttttactttcttttctctcaacatcctccaaacatagccttaatgttgaatatttgttttttttttacatgacgaataccccttgttcttttctcactatctaataaattttattgttaaaaaaaaagattagaatggttttcattctctttttggtcatattgtattccaatttttggatttcactttaccctttaagttttttatttcgtttcattttagttattaggttataaaaaaaaaatcgttctatcatgatctcttaacttatatttatgatggtggaaagtatatccattacaaatactaataaggttaatttagtttggcagcatccttcattttttaatctgtgtaaatatctaagggattaatcaactcttccacatgcagttataatatggtgagtgtaactaaatgaagccttatccattttacactttcataatcacaaagttcttctctccacaaacaccctcatactactcacacaaaggacgtgtcatctcataaatcacaaaacgacaacattactgtcggtactcaataaaacacacccaacacgtatccctgtacgtggcgatacaatgttaacacgtgtcagatatttgcaaaatccaaattccaaaccaatttcaccgaccccatcttaagatattttttcatgtcacatatacttagatttttgcctataaaaacacgacaagttccactaccataaccacatcaaaacacagtctctttacatacactacatttagataaaaaaaaaaactttggaaacaagaaaacaaaacttcacgttatttttggtttgtgaagaagaataaaataacaatgtctcaatatcaacaaggtgg

SEQ ID NO: 2 - DNA, 1333 bp, Artificial Sequence
  Description of Artificial Sequence Synthetic polynucleotide
ggataagattaaggagaagcttcccggtactggatcatgtactggacatggacaaactaaaccataatgtgtgtatatggatgcatgcatttgatatgatttgatatgatcaatcagtagaataaatatgtgtgtattggtgtattttaaacttgtttttgagtcatgttgtttcatatgtatcatatcttatcaccacctcatgtaagagatatatatgaggtgtataatgatgggatatattgattaataagatatgtatgttgtattagtgtaggggccatatgtaaagttactttgaaatagaagtttctttcattgaaaaagtaataaaagttataagtaccaattcctatttcttagaacataacataaacttcagggtttaaagccatgaatctatctttccaagtatattatgaaaaattatcactagaatatatgatcgtttacaaaatttagtgttattctaaagattcaaaactggcaatttgttgtgtagcgaactctatggttgaggggggtcatttctctctctttctatcgatccaaacatgtttctttcatttggtttttgggggctagtacttggtttctgttcttcaatattttcagcattgttttcactcaaatcctgtatgtccaatatctcaactcttcgatttgtttttttctttatcttgattgcaacatcttgagggatgaaacttccacacacaattaccctggtttgcttcttctctaagaaatggctctccaactctagaagcaattaagatcaaagaaacaaataagattatttatattgtacataaatatatttttttataacaaatattagtaataatatgttttttttttggggaaaatatataaaagaaaattatacataaggggaagacaagcggactgtgtcgttaagctttttggaccgtaaacacgacatccataaacccaaaatacatatttgttatgtatgactgtttgaacttaatgttagtaatgtttttttctcctttgtctagatttgaactcaagatctccaacttcttatatgttaacttaaatcagttaagttacccaaccttctattatacatactttttaatttcaaaacgatttatggatataattacttgatagtattaagaaaatgatactagaaagataaaaaggaacattcttcgagcacactaaagatanggttaaggaattgaggtnagataatttcngtccnagaatttgccggagggctgaaggagtgtgaggttgaaacctcnctaaggcganttcnactaccagncgggggagaaacagggtgcggaangggcga

SEQ ID NO: 3 - DNA, 26 bp, Artificial Sequence
  Description of Artificial Sequence Synthetic primer
ccatattcatcaaccctaccagtttg

SEQ ID NO: 4 - DNA, 25 bp, Artificial Sequence
  Description of Artificial Sequence Synthetic primer
ccaccttgttgatattgagacattg

SEQ ID NO: 5 - DNA, 27 bp, Artificial Sequence
  Description of Artificial Sequence Synthetic primer
gacatgatgagcaacaccttggtgaga

SEQ ID NO: 6 - DNA, 28 bp, Artificial Sequence
  Description of Artificial Sequence Synthetic primer
ggataagattaaggagaagcttcccggt

SEQ ID NO: 7 - DNA, 46 bp, Artificial Sequence
  Description of Artificial Sequence Synthetic primer
gtttctacaggacgtaacattgttattttattcttcttcacaaacc

SEQ ID NO: 8 - DNA, 21 bp, Artificial Sequence
  Description of Artificial Sequence Synthetic primer
atgttacgtcctgtagaaacc

SEQ ID NO: 9 - DNA, 24 bp, Artificial Sequence
  Description of Artificial Sequence Synthetic primer
tcggtataaagacttcgcgctgat

SEQ ID NO: 10 - DNA, 27 bp, Artificial Sequence
  Description of Artificial Sequence Synthetic primer
tgcagagctctgtgtgtatatggatgc

SEQ ID NO: 11 - DNA, 30 bp, Artificial Sequence
  Description of Artificial Sequence Synthetic primer
agctgaattccacagtccgcttgtcttccc

SEQ ID NO: 12 - DNA, 21 bp, Artificial Sequence
  Description of Artificial Sequence Synthetic primer
cttctctccacaaacaccctc

SEQ ID NO: 13 - DNA, 43 bp, Artificial Sequence
  Description of Artificial Sequence Synthetic primer
gcaaggtccacacccaagccattgttattttattcttcttcac

SEQ ID NO: 14 - DNA, 22 bp, Artificial Sequence
  Description of Artificial Sequence Synthetic primer
atggcttgggtgtggaccttgc

SEQ ID NO: 15 - DNA, 30 bp, Artificial Sequence
  Description of Artificial Sequence Synthetic primer
ataagagctctcatttaccaggagagtggg

SEQ ID NO: 16 - DNA, 47 bp, Artificial Sequence
  Description of Artificial Sequence Synthetic primer
ccaagtatctgaggtgtgaaaaccattgttattttattcttcttcac

SEQ ID NO: 17 - DNA, 26 bp, Artificial Sequence
  Description of Artificial Sequence Synthetic primer
atggttttcacacctcagatacttgg

SEQ ID NO: 18 - DNA, 33 bp, Artificial Sequence
  Description of Artificial Sequence Synthetic primer
atatgagctcctaacactcattcctgttgaagc

SEQ ID NO: 19 - DNA, 1420 bp, Artificial Sequence
  Description of Artificial Sequence Synthetic polynucleotide
actatagggcacgcgtggtcgacggcccgggctggtatcaaagataaaacatatgcattggtatgtgtgttgaagtcaactatgtcatctattgagggacggagggaatagaaaagaaagacatgcgggaccccactacaaaatgtttccacccacttctgtgcagaaaggttgggaaagcgctgcatttccatctttacccatataaataaattatataatatggtattatgtataatactattatatatcacttaaaagttaaaagttaaaacaagtatagtattatatgtggtaaacagtatagtattgtatagtatatctacaatcaaatattcactctcatgtatactttaacatatcaatttttatttattaataggtaaatattaatatattatattaaataaataataaatatataacccattttatcatagggatatttatgtcatttacaaaatatacatctttctttcctttcactttttcacttattccaaacaatccaaaaaatcatcttactttcctctcactttcattccttaatcattctttccttttactttcttttctctcaacatcctccaaacatagccttaatgttgaatatttgttttttttttacatgacgaataccccttgttcttttctcactatctaataaattttattgttaaaaaaaaagattagaatggttttcattctctttttggtcatattgtattccaatttttggatttcactttaccctttaagttttttatttcgtttcattttagttattaggttataaaaaaaaaatcgttctatcatgatctcttaacttatatttatgatggtggaaagtatatccattacaaatactaataaggttaatttagtttggcagcatccttcattttttaatctgtgtaaatatctaagggattaatcaactcttccacatgcagttataatatggtgagtgtaactaaatgaagccttatccattttacactttcataatcacaaagttcttctctccacaaacaccctcatactactcacacaaaggacgtgtcatctcataaatcacaaaacgacaacattactgtcggtactcaataaaacacacccaacacgtatccctgtacgtggcgatacaatgttaacacgtgtcagatatttgcaaaatccaaattccaaaccaatttcaccgaccccatcttaagatattttttcatgtcacatatacttagatttttgcctataaaaacacgacaagttccactaccataaccacatcaaaacacagtctctttacatacactacatttagataaaaaaaaaaactttggaaacaagaaaacaaaacttcacgttatttttggtttgtgaagaagaataaaataacaatgtctcaatatcaacaaggtgg

SEQ ID NO: 20 - DNA, 1333 bp, Artificial Sequence; identical to SEQ ID NO: 2
  Description of Artificial Sequence Synthetic polynucleotide